(19) United States
(12) Patent Application Publication
(10) Pub. No.: US 2004/0103561 A1
(43) Pub. Date: Jun. 3, 2004

(54) Footwear with orthopedic component system

(76) Inventors: Todd D. Campbell, Petaluma, CA (US); Russell C. Davis, Greenbrae, CA (US); William Y. Guthrie, Fairfax, CA (US)

Correspondence Address: JOHNSON & STAINBROOK, LLP, 3550 Round Barn Blvd., Suite 203, Santa Rosa, CA, 95403, US

(21) Appl. No.: 10/722,088

(22) Filed: Nov. 24, 2003

Related U.S. Application Data
(63) Continuation-in-part of application No. 10/136,770, filed on Apr. 30, 2002.
(60) Provisional application No. 60/288,319, filed on May 2, 2001.

(51) Int. Cl. A43B 7/14; A61F 5/14

(52) U.S. Cl. 36/88; 36/92; 36/155

(57) ABSTRACT

The invention provides an innersole system for an article of footwear, including an orthopedic intermediary support member and a user-selectable exchangeable innersole. The orthopedic intermediary support member includes a cupped heel portion having a concave upper bearing surface and a midfoot portion having a medial longitudinal arch support with a curvilinear upper bearing surface. The exchangeable innersole mates with the orthopedic intermediary support member to provide a user-selected orthopedic correction factor.

### CROSS REFERENCE TO RELATED APPLICATIONS

This utility application is a continuation-in-part of currently pending U.S. patent application Ser. No. 10/136,770 filed Apr. 30, 2002. The present application claims the benefit of the filing date of U.S. Provisional Application Serial No. 60/288,319, filed May 2, 2001.

### STATEMENT REGARDING FEDERALLY SPONSORED RESEARCH OR DEVELOPMENT

Not applicable.

### REFERENCE TO A MICROFICHE APPENDIX

Not applicable.

### TECHNICAL FIELD

The present invention relates generally to footwear with orthopedic devices, and more particularly to a multi-innersole component system for an article of footwear that helps abate and prevent foot and related ailments.

### BACKGROUND INFORMATION AND DISCUSSION OF RELATED ART

Custom orthotics for feet typically contain a relatively rigid, resilient base comprising a heel portion and an arch portion, contoured to fit the plantar or bottom surface of the foot. Orthotic devices may be inserted into footwear to reduce pronation of feet and to provide a therapeutic and corrective effect for foot ailments such as plantar fasciitis, cuboid syndrome and tissue trauma. Custom-made orthotics are generally created from hard plastics by using a mold and extensive measurements of an individual's foot, and modified as needed to provide prescribed corrections by a podiatrist. Unfortunately, custom orthotics typically fit into only one or a few pairs of shoes, and are too expensive for a wide variety of shoes that might be part of a person's wardrobe. Because of the expense, orthotic devices are often used only after serious degradations of a foot problem and severe increase in foot pain. As a foot condition improves, prescribed orthotics may require alterations with less correction or be discarded altogether. Although highly beneficial in correcting early onsets of podiatric conditions, custom orthotics for a child may be considered cost prohibitive, with frequent size alternations needed due to rapid growth of the feet and changing body physiology of the child.

In contrast to custom orthotics, inexpensive shoe inserts, which often comprise relatively thin layers of foam material, can be removed from shoes so that they may be washed, replaced, or used in other shoes. These inserts may provide some additional comfort, albeit with temporary relief and minimal physiological effect.

A conventional insole often consists of materials such as elastomeric foam that are covered with and adhesively bonded to leather, natural fabrics or other synthetic materials. The insole may be contoured to conform to the outline of the foot for placement atop the sole of a shoe. The insoles generally have a flat shape cut out of a sheet of material in the shape of the foot, with additional material in the area corresponding to the arch portion of the foot, and more material in the region surrounding the heel of the foot. Many conventional insoles use an impact cushioning layer or space filler made from synthetic polyurethane or polyolefin foam that has compression-deformation characteristics. Thus, when a wearer puts on a shoe having such an insole, the impact cushion layer of the insole is deformed with the weight of the wearer and conforms to the shape of the foot. Over time and with use, these deformations may become permanent, forfeiting the initial comfort and impact-absorbing qualities of the insole.

While commonly used, conventional insoles are often ineffective in preventing or alleviating pain associated with foot conditions such as plantar fasciitis. The top surface of their contoured foam material are designed to support and cradle the foot, but the foam is intended mainly to cushion the foot and not to provide the necessary support for proper biomechanical functions of the foot, particularly in the rear foot and arch areas. Non-custom accommodative orthotics tend to be fabricated from a soft material that compresses under loads, so as to be tolerated by a wide variety and shapes of feet. While increasing foot comfort, they are unlikely to provide significant control of foot motion.

Some over-the-counter insoles provide limited support in the heel and rear foot areas, but do little to prevent excessive foot pronation. Pronation is an inwardly declining complex motion of the calcaneous or heel bone involving a partial collapse of the medial longitudinal arch of the foot. It occurs during ambulation, after an initial heel strike as the weight and movement of the body progresses forward onto the balls of the feet.

Some recent efforts have been made to correct foot problems with firmer and higher durometer materials added to the arch area and around the heel of the foot. These devices, most of them custom-molded for the foot, are designed to resist pronation and to distribute weight-bearing stresses to areas of the foot that can better tolerate such stresses, thus maximizing comfort and minimizing trauma to the sole of the foot. Such an orthotic device provides a padded surface that is shaped to conform to the contours of a particular foot. A corrective orthotic may be designed to guide and restrict the motion of joints of the foot in order to improve gait efficiency and to reduce the stresses imposed on lower extremity anatomical structures during walking, running and standing.

An exemplary and painful foot condition for which orthotic devices are often used is plantar fasciitis. Extended flattening or stretching of the plantar fascia, and secondarily from microscopic tears and tissue irritation resulting from such flattening or stretching, primarily cause plantar fasciitis. Its etiology may include a traumatic event or sustained trauma from ambulatory actions, or it may be due to very mild congenital foot malformations such as flat feet or high arches. Plantar fasciitis is an inflammation of the plantar fascia, which encapsulates muscles in the bottom of the foot and supports the arch of the foot by acting as a bowstring that connects the balls of the foot to the heel. The plantar fascia endures tensional forces that are approximately twice the body weight during walking at the moment when the heel of the trailing leg begins to lift off the ground. This moment of maximum tension is increased and intensified suddenly, particularly when there is lack of flexibility in the calf muscles. A percentage increase in body weight causes the same percentage increase in tension in the fascia. Due to the repetitive nature of walking, plantar fasciitis may be a repetitive stress disorder (RSD) not unlike carpal tunnel syndrome and tennis elbow. All three conditions benefit greatly from rest, ice, and periodic stretching, but may also be treated with non-steroidal anti-inflammation pills (NSAIDs), mechanical splints or straps, and as a last resort, surgery. Other biomechanical and other non-surgical methods for treating plantar fasciitis include injecting steroids, limiting heel strikes, and using heel cups, cushioned inserts, shock-absorbing athletic shoes, crepe-soled shoes, aspirin, a short leg walking cast, heat, ultrasonic treatment and custom orthotics. These treatments and their effectiveness remain unpredictable, sometimes requiring years for foot problems to abate.

Another treatment for plantar fasciitis, as well as other related foot conditions, is the taping of a foot so as to reduce subtalar joint motion and thereby restrict pronation. This may provide immediate pain relief and the reduction of irritation so that a person may better tolerate a brief period of time while customized orthotic devices are shaped and delivered. However, taping requires a considerable investment in time and requires the expertise of an orthopedist, therapist or trainer specifically skilled in the art. Additionally, taping provides relief and therapeutic efficacy for only a few days, as the tape invariably loosens. Taping may impact normal activities, such as bathing or the selection of footwear. Accordingly, it would be desirable to provide an effective orthopedic device without the need for taping or any other time-consuming task.

Conditions that may benefit from similar orthopedic devices to those used for plantar fasciitis are arthritis, heel bone damage, bone spurs, stress fractures, loss of natural tissue for cushioning under the heel (“fat pad atrophy”), tarsal tunnel syndrome (the foot's version of carpal tunnel syndrome), stress fractures, tendinitis, and complications from diabetes.

Many people may benefit from non-custom orthotic devices and not require expensive, individually fabricated orthotic devices. A desirable orthopedic device, which provides an alternative to some custom orthotics prescribed by medical specialists, cooperatively redistributes the normally greater weight-generated forces applied to the inner and more bony regions of the heel outwardly toward the outer and more fleshy regions of the heel. Additionally, it would provide support and stability to affected areas of the foot and reduce subtalar joint motion. The beneficial orthotic device would also have an ability to adjust for variations in gait, foot and shoe size without the need for a custom fit. In this respect, the adaptability of the devices may lower the expense and limit the need for medical assistance in prescribing and fitting the devices.

Furthermore, podiatrists and other medical practitioners would benefit from being able to provide effective non-custom orthotic devices for their patients while they wait the usual several weeks to receive custom-built orthotics. These specialists also would be able to provide a line of shoes with a built-in orthopedic device that allows the patient to select from immediately available, off-the-shelf shoes offering therapeutic properties.

A desirable orthotic device is adaptable to many types and sizes of adult and children's shoes, integrated into the insole or secured onto the sole of a shoe. While orthotic devices are generally considered to be therapeutic and restorative, a beneficial foot orthotic would help prevent foot problems caused by pronation, excessive motion of joints, and increased stresses on vulnerable areas of a foot.

Therefore, an object of this invention is to provide an orthopedic system for footwear that provides preventative and curative properties for a variety of foot ailments and that overcomes the deficiencies and obstacles described above.

### BRIEF SUMMARY OF THE INVENTION

One aspect of the invention is an innersole system for an article of footwear including an orthopedic intermediary support member and a user-selectable exchangeable innersole. The orthopedic intermediary support member includes a cupped heel portion having a concave upper bearing surface and a midfoot portion having a medial longitudinal arch support with a curvilinear upper bearing surface. The exchangeable innersole mates with the orthopedic intermediary support member to provide a user-selected orthopedic correction factor.

Another aspect of the invention is an article of footwear, including an orthopedic intermediary support member and a user-selectable exchangeable innersole. The orthopedic intermediary support member is fixedly attached to the article of footwear, and includes a cupped heel portion having a concave upper bearing surface and a midfoot portion having a medial longitudinal arch support with a curvilinear upper bearing surface. The exchangeable innersole mates with the orthopedic intermediary support member to provide a user-selected orthopedic correction factor.

Another aspect of the invention is a method of using a multi-component orthopedic system. An article of footwear having an orthopedic intermediary support member fixedly attached to the article of footwear is provided. An orthopedic condition of a user is determined. One innersole is selected from a set of exchangeable innersoles that mates with the orthopedic intermediary support member to provide an orthopedic correction factor. The selected exchangeable innersole is inserted into the article of footwear.

The aforementioned, and other features and advantages of the invention will become further apparent from the following detailed description of the presently preferred embodiments, read in conjunction with the accompanying drawings. The detailed description and drawings are merely illustrative of the invention rather than limiting, the scope of the invention being defined by the appended claims and equivalents thereof.

### BRIEF DESCRIPTION OF THE SEVERAL VIEWS OF THE DRAWINGS

The following drawings are shown with right feet, right orthopedic intermediary support members, and right shoes, and it should be understood that the left foot, orthopedic intermediary support members, exchangeable innersoles, and shoes are substantially mirror images of the right side. It should also be understood that the use of the word shoe, in the context of this document, is intended to be synonymous with nearly all articles of footwear, including but not limited to boots, sandals, open-toe shoes and closed-toe shoes. Further characteristics and advantages of the invention will become apparent from the following detailed descriptions of particular but not exclusive embodiments, illustrated by way of non-limiting examples in the accompanying drawings, wherein:

FIG. 1 illustrates a side view and a top view of a human foot;

FIG. 2 illustrates a perspective view of an innersole system for an article of footwear, in accordance with one embodiment of the current invention;

FIG. 3 illustrates a top view of an orthopedic intermediary support member extending from the heel to the balls of a foot, in accordance with one embodiment of the current invention;

FIG. 4a illustrates a cross-sectional view of an orthopedic intermediary support member, in accordance with one embodiment of the current invention;

FIG. 4b illustrates a perspective view of an orthopedic intermediary support member, in accordance with one embodiment of the current invention;

FIG. 5 illustrates a bottom view of an exchangeable innersole for a pronation condition, in accordance with one embodiment of the current invention;

FIG. 6 illustrates a bottom view of an exchangeable innersole for a supination condition, in accordance with one embodiment of the current invention;

FIG. 7 illustrates a bottom view of an exchangeable innersole for a neutral condition, in accordance with one embodiment of the current invention;

FIG. 8 illustrates a perspective view of an orthopedic intermediary support member mated with an exchangeable innersole, in accordance with one embodiment of the current invention;

FIG. 9 illustrates a perspective view of shoe with an orthopedic intermediary support member and a user-selectable exchangeable innersole, in accordance with one embodiment of the current invention;

FIG. 10 illustrates a perspective view of boot with an orthopedic intermediary support member and a user-selectable exchangeable innersole, in accordance with one embodiment of the current invention;

FIG. 11 illustrates a flow diagram of a method for using a multi-component orthopedic system, in accordance with one embodiment of the current invention; and

FIG. 12 illustrates a flow diagram of a method for manufacturing an article of footwear including an orthopedic intermediary support member and an exchangeable innersole, in accordance with one embodiment of the current invention.

### DETAILED DESCRIPTION OF THE INVENTION

The multi-component orthopedic system of the present invention can benefit the fitness of the foot by reducing subtalar joint motion, limiting the stretching of the plantar fascia and stabilizing the heel of the foot while walking or running. The present invention assists in inverting the subtalar joint to a position of slight inversion and simultaneously, plantar-flexing the first ray to lock the midtarsal joint during ambulation. Thus, the present invention reduces excessive pronation, a condition that often leads to foot injury. The therapeutic device is capable of relieving foot pain and biomechanically correcting or alleviating misaligned conditions in a foot. The device may help prevent or provide relief from common foot problems such as heel spurs, arch pain, metatarsalgia (ball-of-foot pain), bunions, hammertoe, arthritis, neuromas, diabetes foot, plantar fasciitis, cuboid syndrome, tendinitis, stress fractures, shin splints, and other ailments of the foot, leg, and lower back. Diabetics, for example, may be more susceptible to foot disease such as ulcers or sores caused by infection and minor injuries that may be avoided or corrected by use of an effective device. Although the invention may serve as an aid in the recovery from a foot ailment, the invention may also serve to prevent the onset or reoccurrence of various foot problems and athletic injuries.

The multi-component orthopedic system includes an orthopedic intermediary support member and a user-selectable exchangeable innersole. The orthopedic intermediary support member includes a high-rise heel cup portion that along with the exchangeable innersole absorbs shock during heel strikes, while providing support to the proximal, distal and posterior of the calcaneous. The orthopedic intermediary support member includes a midfoot portion that cooperates with the heel portion to stabilize and support the foot while preventing excessive pronation, and provides a therapeutic characteristic for a podiatric condition such as plantar fasciitis.

The deep heel cup portion of the present invention wraps around the heel of the foot and extends above a posterior portion of the heel bone proximal to the Achilles tendon. The midfoot portion of the orthopedic intermediary support member is continuously coupled to the cupped heel portion. The midfoot portion includes a curvilinear upper bearing surface and a medial longitudinal arch support to aid in the support of the medial longitudinal arch of the foot. The upper surface of the midfoot portion may include a minor arch to support the lateral longitudinal arch near the outside of the foot, and a minor arch to support the transverse arch perpendicular to the medial longitudinal arch and the lateral longitudinal arch of the foot. The midfoot portion extends from the heel cup towards the heads or anterior ends of the metatarsal bones.

The orthopedic intermediary support member may also include a forefoot portion that is a generally flat or planar section continuously coupled to the midfoot portion, and contoured around the perimeter to correspond to the inside of a shoe.

The cupped heel portion and the midfoot portion cooperate to help realign the rear foot to avoid overpronation and reduce stress on the Achilles tendon. Excessive pronation renders the gait of a walker or runner less efficient, and is a source of lower extremity pathologies, including muscle tiredness and inflammation, foot and knee joint pain, tendinitis, ligament strain, and even neurological damage. When the orthopedic intermediary support member and the exchangeable innersole are designed for the full length of the foot, the forefoot portion of the component system cushions and reduces stress on the balls and phalangeal area of the foot.

The orthopedic intermediary support member is integrated into or attached to an inner surface of the shoe. The actual dimensions of an orthopedic intermediary support member, in accordance with the present invention, will vary depending on the length and width of the foot, the intended use of the shoe, and other factors. The net result of various embodiments of the present invention is a multi-innersole component system that controls pronation, supports the foot, and produces a more stable platform on which the foot ambulates.

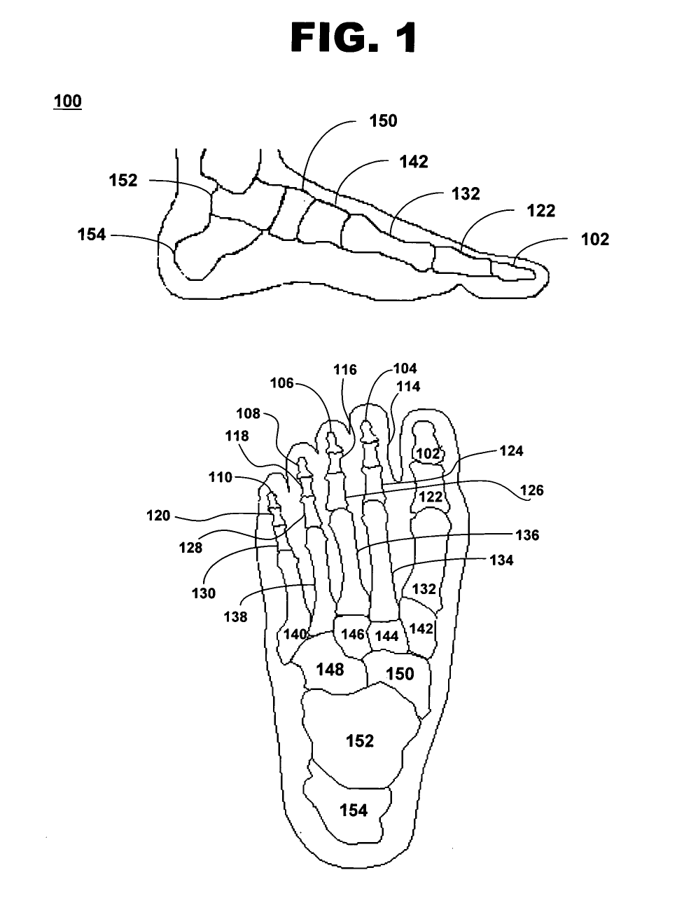

FIG. 1 illustrates a side view and a top view of a human foot at 100. The toes of a human foot are formed by fourteen phalanges. Starting from the inside of the foot, each toe has distal phalanges 102, 104, 106, 108 and 110, middle phalanges 114, 116, 118 and 120, and proximal phalanges 122, 124, 126, 128 and 130. The first phalange or big toe lacks a middle phalange. The forefoot comprises the phalanges and the heads or anterior end of the metatarsals.

The midfoot includes five metatarsals 132, 134, 136, 138 and 140. First metatarsal 132, which is the shortest and thickest of the metatarsal bones, bears the most weight and plays the most important role in propulsion. First metatarsal 132 also provides attachment for several tendons. The more stable second metatarsal 134, third metatarsal 136, and fourth metatarsal 138 are well protected with only minor tendon attachments, and thus are not subjected to strong pulling forces.

The midfoot also includes five of seven tarsal bones: navicular, cuboid, and cuneiform bones. The distal row contains three cuneiforms 142, 144 and 146 and a cuboid 148. The midfoot includes five tarsometatarsal joints, which are among multiple joints within the midfoot itself. Proximally, cuneiforms 142, 144 and 146 articulate with a navicular 150.

A talus 152 and a calcaneous 154 make up the rear or hind portion of the foot. Calcaneous 154 is the largest tarsal bone, and forms the heel. Talus 152 rests on top of it, and forms the pivot for the ankle. Toe movements take place at joints that are capable of motion in two directions: plantar flexion and dorsiflexion, as well as abduction and adduction. The remainder of the foot has two movements, inversion and eversion, to which joints of the hindfoot and midfoot contribute. These complex movements are combined ordinarily with ankle movements and movements of the fibula and tibia.

Two primary functions of the foot are weight bearing and propulsion, both requiring stability and flexibility. The bones and intervening joints of the foot give flexibility while multiple bones form an arch to support the weight of the body.

The three arches of the foot are the medial longitudinal arch, lateral longitudinal arch, and transverse arch. The inner or medial longitudinal arch, the highest of the arches, comprises calcaneous 154, talus 152, navicular 150, cuneiforms 142, 144 and 146, and first three metatarsals 132, 134 and 136. The outer or lateral longitudinal arch, which is lower and flatter than the medial arch, comprises calcaneous 154, talus 152, cuboid 148, and fifth metatarsal 140. At times, fourth metatarsal 138 is included in the lateral arch. The generally hemispherical arc of the transverse arch comprises cuneiforms 142, 144 and 146, cuboid 148, and the bases of metatarsals 132, 134, 136, 138 and 140. The arches of the foot are maintained by the shapes of the bones and ligaments, and supported by muscles and tendons. The lateral arch, medial arch and transverse arch aid the foot in supporting and distributing the weight of a person. During a heel strike, for example, the force on the heel region may exceed three times the normal weight of the body.

When walking, body weight is first placed on the heel, then forward to the ball of the foot. As body weight is applied to the foot, the arches flatten out slightly to absorb the added pressure, spreading out the force and strain across the bones of the foot evenly. As the foot is lifted before taking another step, the arch springs back into its arched position.

The foot has two primary motions: supination and pronation. Supination is a combination of inward rotation at the ankle, adduction of the hindfoot, inversion of the forefoot, and medial arch elevation. Supination occurs when a heel comes off the ground. Subtalar joint supination involves three simultaneous planes of motion: adduction, inversion, and plantarflexion. As the foot supinates, lateral structures tighten. Continued supination and adduction force may rupture portions of lateral collateral ligaments or avulse these ligaments from their bony attachment sites on the distal fibula, resulting in an ankle sprain.

Subtalar joint pronation involves three simultaneous planes of motion: abduction of a forefoot, eversion of a hindfoot, and dorsiflexion. Because of the close contiguity of the joints involved, pronation is accompanied by eversion of the heel and internal rotation of the leg and hip. In simple terms, pronation is a motion that occurs when the foot lands on the outside edge and the inner arch collapses as far as it can to absorb shock.

Overpronation, the maximum range of motion between pronation and supination, is often cited as a cause of leg and foot problems among runners and a contributor to knee, hip and back pain. While pronation is a normal part of a person's gait, it is understood that excessive pronation may be the source of many lower extremity pathologies, including muscle tiredness and inflammation, foot and knee joint pain, tendinitis, ligament strain, and even neurological damage. Excessive pronation may render the gait less efficient since time and effort is wasted in pronating and supinating.

Oversupination, which has been estimated to affect 5 to 10% of people with abnormal foot mechanics, occurs when the joints in the ankle excessively supinate and prevent proper foot pronation, thus locking the ankle joints and preventing proper shock absorption. Oversupination is often associated with rigid, high arches. Oversupinators may suffer from problems related to poor shock absorption and are at greater risk for experiencing low back, lateral knee, and hip pain, as well as for developing degenerative arthritis (osteoarthritis) at the major weight bearing joints.

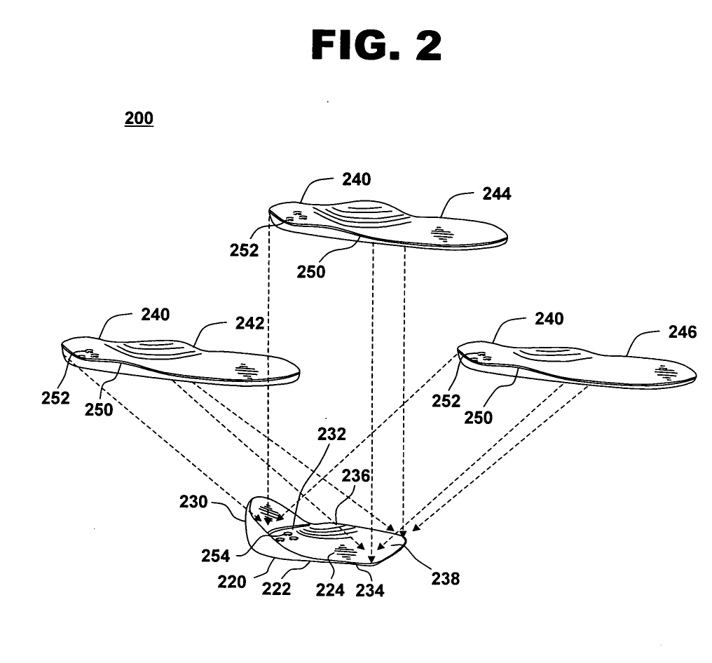

FIG. 2 illustrates a perspective view of an innersole system for an article of footwear, in accordance with the present invention at 200. Innersole system 200 includes an orthopedic intermediary support member 220 and one or more user-selectable exchangeable socks or innersoles 240. Orthopedic intermediary support member 220 includes a cupped heel portion 230 having a concave upper bearing surface 232 and an upwardly concave shape for engaging the heel of a foot, and an upwardly arched midfoot portion 234 having a medial longitudinal arch support 236 with a curvilinear upper bearing surface 238 for engaging the arched portion of a foot. Exchangeable innersole 240 mates with orthopedic intermediary support member 220 to provide a user-selected orthopedic correction factor such as a pronation correction factor, a supination correction factor, and a neutral correction factor. Cupped heel portion 230 and midfoot portion 234 of orthopedic intermediary support member 220 cooperate with exchangeable innersole 240 to invert a subtalor joint of a foot to a position of slight inversion to lock a midtarsal joint during ambulation of the foot.

Exchangeable innersoles 240 may comprise a set of exchangeable innersoles including, for example, a pronation innersole 242, a supination innersole 244, and a neutral innersole 246 that a user may select from and that supports most foot types comfortably and correctly. Multiple exchangeable innersoles provide biomechanical support for pronators, supinators, and those with other foot variations. Orthopedic intermediary support member 220 and exchangeable innersoles 240 offer foot support with their biomechanical heel positing and arch support. Each exchangeable innersole 240 includes a built-up medial arch support based on an orthopedic correction factor. Thickness variations and use of more rigid materials in the innersole region corresponding to the medial longitudinal arch provide for various orthopedic correction factors. Exchangeable innersoles 240 and orthopedic intermediary support member 220 cooperate to provide correction for a pronation condition or a supination condition. Although not shown in this figure, orthopedic intermediary support member 220 is fixedly attached to an article of footwear. A user inserts exchangeable innersoles 240 into a shoe.

Orthopedic intermediary support member 220 has midfoot portion 234 formed continuously with cupped heel portion 230. Midfoot portion 234 extends from cupped heel portion 230 to an opposite end corresponding to the anterior ends of the metatarsal bones, and from the inner or medial portion to the outer or lateral side of the foot. This two-thirds or partial length orthopedic intermediary support member 220 extends from the heel to the balls of a foot. Points near the medial end of midfoot portion 234 may be more forward than points near the lateral end of midfoot portion 234. The front edge of the partial-length orthopedic intermediary support member may be straight or curvilinear. The thickness of midfoot portion 234 may diminish to a smooth taper at the front edge. The lower bearing surface of cupped heel portion 230 and midfoot portion 234 are essentially flat or may be shaped to substantially conform to an inside surface of a shoe.

A longer, full-length version of the support member extends through the anterior end of the foot. Therapeutic and preventative aspects of the partial-length orthopedic intermediary support member are retained in the full-length embodiment.

Cupped heel portion 230 extends above a most posterior cephalad portion of a calcaneous and is continuously coupled to midfoot portion 234. Frontal extremities of cupped heel portion 230 may be positioned somewhat more forwardly on the medial side than on the lateral side. Cupped heel portion 230 deforms to conform to the shape of the heel and to provide medial, posterior and lateral support to the calcaneous. A posterior surface of cupped heel portion 230 may engage the heel above the heel bone close to the Achilles tendon. A medial surface and a lateral surface of cupped heel portion 230 may engage the heel bone below the ankle malleolus. The upper edge of cupped heel portion 230 extends along an arcuate path in a generally descending manner from the Achilles tendon to midfoot portion 234. Upper bearing surface 232 of cupped heel portion 230 and upper bearing surface 238 of midfoot portion 234 may be continuously curvilinear, adapted to follow the contours of the plantar surface of the foot. A raised arch area in midfoot portion 234 provides support for the arches of the foot without collapsing under body weight. Upper bearing surface 232 of cupped heel portion 230 and upper bearing surface 238 of midfoot portion 234 are contoured to engage the plantar surface of a foot. Lower bearing surface 222 of orthopedic intermediary support member 220 is shaped to substantially conform to an inside surface of the sole of a shoe when attached to or integrated with the sole of a shoe. Lower bearing surfaces of exchangeable innersoles 240 are contoured to conformably mate with upper bearing surfaces 232 and 238 of orthopedic intermediary support member 220 and to the inner surfaces of a shoe along the sides and anterior end.

Orthopedic intermediary support member 220 may act simultaneously on the calcaneous and subtalar of the foot. Cupped heel portion 230 may help to stabilize and control the motion of the foot, keeping the heel in its natural state and preventing it from excessively pronating or rolling inward during walking and running, thereby properly aligning the foot and providing better shock absorption and stress distribution.

Cupped heel portion 230 and midfoot portion 234 cooperate to provide a therapeutic characteristic for a podiatric condition, which may include plantar fasciitis or another medical condition such as cuboid syndrome, a neuroma, hammertoe, a bunion, a pronation condition, tendinitis, or a foot ailment. Other podiatric conditions may include fat pad atrophy, heel spurs, metatarsalgia, diabetic foot, hyperkeratosis, Morton's neuroma, plantar pain from arthritis or peak shock load, sore heels, sore knees, shin splints, Sever's disease, calcaneal apophysitis, bursitis, Achilles tendinitis, and elongated metatarsals.

In an alternative embodiment, a forefoot portion of orthopedic intermediary support member 220 extends from the forward end of midfoot portion 234 to an end of orthopedic intermediary support member 220 corresponding to the metatarsal heads of a wearer's foot, and from a medial side to a lateral side of the foot. The forefoot portion of orthopedic intermediary support member 220 may have a relatively thin, substantially planar upper bearing surface. The forefoot portion is continuously coupled to midfoot portion 234 and extends from the front of midfoot portion 234 to a region corresponding with the distal end of the foot while comfortably encompassing the bottoms of the toes. The forefoot portion may reduce stress on the balls of the foot, and aid in distributing ambulatory stresses into the front portion of the foot.

An exemplary embodiment of orthopedic intermediary support member 220 is relatively thick in cupped heel portion 230 under and around the heel of the foot, and relatively thin and flexible near its upper and lateral edges. Orthopedic intermediary support member 220 is relatively thick at the arched regions of midfoot portion 234, particularly in the region under the medial longitudinal arch of the foot, and relatively thin near the sides. In the full-length version, orthopedic intermediary support member 220 is relatively thin and generally flat or planar in the forefoot portion. The thickness and dimensions of various portions or orthopedic intermediary support member 220 and exchangeable innersole 240 may be selected to provide suitable support and stability while thin where possible to maintain low weight and to allow comfortable incursion of a foot into a shoe fitted with orthotic orthopedic intermediary support member 220 and exchangeable innersole 240. The dimensions of orthopedic intermediary support member 220 and exchangeable innersole 240 can be selected to fit snugly into a children's shoe or one of a variety of adult specialty shoes. Orthopedic intermediary support member 220 may have a seamless surface, or may have ribs, contours or cavities to remain lightweight while retaining structural stability.

Material comprising orthopedic intermediary support member 220 provides dynamic control as well as static balance. Orthopedic intermediary support member 220 is made from a semi-flexible material that can cushion and absorb the shock from heel strike on orthopedic intermediary support member 220. Orthopedic intermediary support member 220 comprises a substantially flexible, resiliently compressible cushioning material having an upper surface for engaging a plantar surface of a foot and a bottom surface for engaging a sole of a shoe. Orthopedic intermediary support member 220 may be comprised of a semi-rigid, injection moldable material. The durometer value of the flexible material may extend from a value less than 20 to a value in excess of 70. The flexible material includes, for example, a neoprene rubber, a silicone rubber, an elastomer, a polymeric material, a urethane, polyethylene teraphthalate, a viscoelastic polymer, a silicone gel, or combinations thereof.

Exemplary material used in the orthotic system of the present invention is a compression-resistant, deformable material that provides shock attenuation and support for the foot without use of rigid materials such as posting frequently used in custom orthotic devices. The material may comprise a gripping characteristic to allow the orthopedic intermediary support member to firmly engage a heel bone and provide proximal, posterior and lateral support. Orthopedic intermediary support member 220 may have a texture 224 embossed on upper bearing surfaces 232 and 238 or on lower bearing surface 222 to improve the gripping characteristic. Textured surfaces aid in enhancing the gripping capability of the heel cup to effectively engage the heel and redistribute stresses and to enhance contact with the foot or the sole of a shoe. Textured surfaces such as deep waffle or honeycomb patterns, particularly on lower bearing surface 222, may enhance shock-absorbing qualities of orthopedic intermediary support member 220, or be cosmetic in nature.

Exchangeable innersole 240 may include at least one protrusion 252 such as a peg, a ring, a letter, or other locking feature on a lower bearing surface of exchangeable innersole 240 that locks into a corresponding recess 254 on concave upper bearing surface 232 of orthopedic intermediary support member 220. Ribs, contours or cavities may be formed on or within orthopedic intermediary support member 220 to decrease weight while retaining stability.

In alternative configurations, reinforcing support members may be built into orthopedic intermediary support member 220. For example, a rim region of harder material may surround the base of the cupped heel portion. Reinforcing support members may be built into the cupped heel portion of the orthopedic intermediary support member to provide additional support of the calcaneous, using, for example, semi-circular rods of high strength, resilient material extending around the back and sides of the heel, or upwards from the base of the cupped heel portion towards the ankle. Regions of soft, gel-like material may be incorporated into select regions of the orthopedic intermediary support member, such as directly underneath the fat pad of the foot where heels may bruise and bone spurs may occur. Features such as holes and recesses to lighten the orthopedic intermediary support member in select areas may be included.

The flexible and shock-absorbing polymeric material of exchangeable innersole 240 may be a lightweight and durable thermoplastic such as polyethylene or cross-linked ethylene vinyl acetate foam, cross-linked polyethylene, poly(ethylene-vinyl acetate), polyvinyl chloride, an acrylic, synthetic and natural latex rubbers, block polymer elastomers, thermoplastic elastomers, polystyrene, ethylenepropolene rubbers, silicone elastomers, polystyrene, polyurea or polyurethane, a polyurethane foam, an elastomeric foam, a moldable foam, a non-foam elastomer, a silicone gel, a viscoelastic material, and combinations thereof. The surfaces of the orthopedic intermediary support member may be smooth or embellished with various patterns and textures.

A thin, absorptive layer 250 may be disposed on an upper bearing surface of exchangeable innersole 240 to provide additional comfort for the user and to provide shock absorbing and body moisture absorbing characteristics. Absorptive layer 250 may wick moisture from the foot and allow exchangeable innersole 240 to breathe. Absorptive layer 250 is attached to upper bearing surface of exchangeable innersole 240. Absorptive layer 250 comprises, for example, a compressible polymeric foam with a nominally thickness of one-sixteenth to one-eighth inch. Absorptive layer 250 may be treated with bacteria and fungus inhibitors or odor preventative agents to reduce foot odors. Absorptive layer 250 may include a cloth, polymeric, synthetic or natural leather top layer that is fixedly superposed onto the upper surface of exchangeable innersole 240.

Exchangeable innersole 240 may be covered or partially covered with a top layer such as leather, woven fabrics, unwoven fabrics, or other materials adhesively bonded thereto.

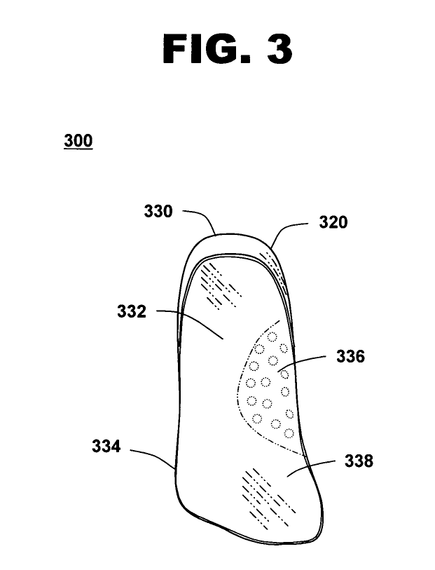

FIG. 3 illustrates a top view of an orthopedic intermediary support member, in accordance with the present invention at 300. In this embodiment, orthopedic intermediary support member 320 is configured with a cupped heel portion 330 and a midfoot portion 334 as a single unit, closed about the heel, extending from molleolar height, proximally, and extending in a contoured fashion distally to the plantar proximal contact surface. Cupped heel portion 330 and midfoot portion 334 are continuously connected and adapted to form around and engage the heel of a foot and to support the arches. Cupped heel portion 330 comprises an upwardly concave upper bearing surface extending from beneath the heel to a point above the calcaneous, with a tapered, upper edge generally descending from the back of the heel near the Achilles tendon to midfoot portion 334. Upwardly arched midfoot portion 334 includes a medial longitudinal arch support 336 and a curvilinear upper bearing surface 238 for engaging an arch portion of the foot. An upper bearing surface 332 of cupped heel portion 330 continuously connects with upper bearing surface 338 of midfoot portion 334.

The perimeter of midfoot portion 334 extends from cupped heel portion 330 along the outer contours of the medial longitudinal arch of the foot, traversing laterally underneath the metatarsal bones of the foot, and continuing along the outer contours of the lateral longitudinal arch of the foot to cupped heel portion 330.

Upper bearing surface 338 of midfoot portion 334 is contoured to support the medial longitudinal arch, the lateral longitudinal arch, and the transverse arch of the foot. The lower surfaces of midfoot portion 334 and cupped heel portion 330 are nominally flat or shaped to conform to an inside surface of a shoe.

The orthopedic intermediary support member maintains a subtalor joint in an inverted position, and locks the midtarsal joint during ambulation of the foot. Cupped heel portion 330 may include relieved or cutout areas. Cupped heel portion 330 may be continuously joined to midfoot portion 334 to support the calcaneous and permit limited freedom of movement of the heel relative to the midfoot portion. Cupped heel portion 330, which includes a lower surface extending longitudinally and continuously under midfoot portion 334, is adapted to surround the heel and adjacent portions of a foot. A reinforcing support member may be built into cupped heel portion 330 to provide additional support of the sides, back and bottom portions of the heel, yet retain a cushioning, impact absorbing characteristic under particularly sensitive portions of the heel or foot.

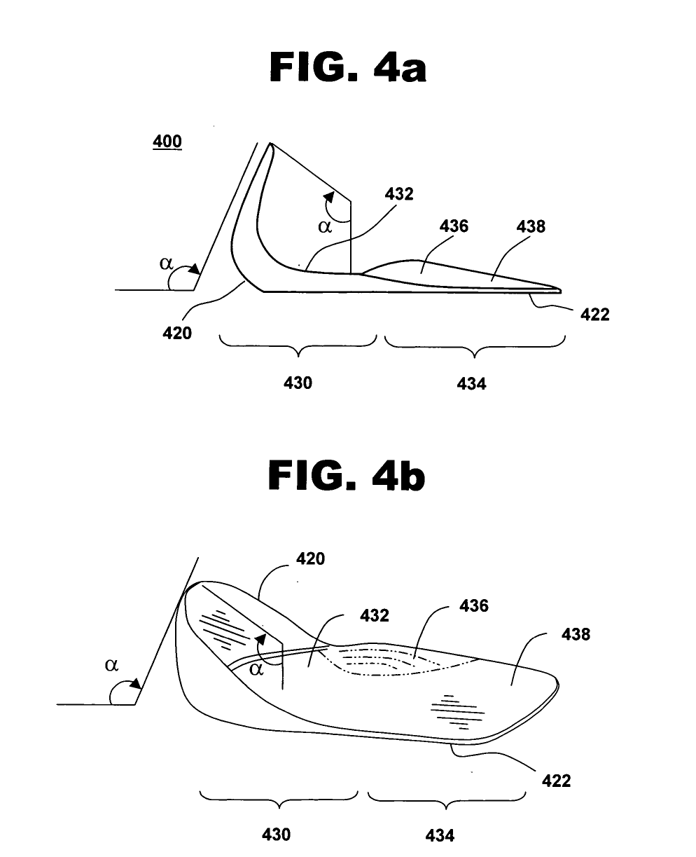

FIG. 4a illustrates a cross-sectional view of an orthopedic intermediary support member, in accordance with the present invention at 400. Orthopedic intermediary support member 420 comprises a cupped heel portion 430 and a midfoot portion 434 with upper bearing surfaces 432 and 438, respectively. Midfoot portion 434 includes a medial longitudinal arch support 436. Orthopedic intermediary support member 420 may comprise an additional forefoot portion continuously coupled to a posterior end of midfoot section 434. The lower bearing surface 422 of the full-length version or two-thirds length version of orthopedic intermediary support member 420 is contoured to conform to an inside surface of a shoe and may have some texture, embossed patterns or other indenting or protruding features, with continuous surfaces that align with the inner surface of a shoe.

The rear part of cupped heel portion 430 opens toward midfoot portion 434, the heel cup being designed and dimensioned for adapting to the calcaneous. Cupped heel portion 430 may be continuously curved. An inner arcuate portion and an outer arcuate portion of cupped heel portion 430 above the calcaneous may be angled forwardly and upwardly and accorded a heel cup angle alpha (α), the heel cup angle alpha being measured by an arc sweeping from the base of the upwardly concave cupped heel portion 430 to the top of the inner arcuate portion. Alternatively, heel cup angle alpha may be measured by an angle corresponding to a line essentially parallel to lower bearing surface 422 of cupped heel portion 430 and a line essentially tangential to the top of the outer arcuate portion, with a larger heel cup angle corresponding to a fuller heel cup. The heel cup angle of the currently preferred embodiment may be greater than 60 degrees, and in one embodiment on the order of 90 degrees.

A larger heel cup angle provides more support and stability for the calcaneous, cooperating with midfoot portion 434 to invert the subtalor joint of a foot to a position of slight inversion while walking or running.

The medial, posterior, and lateral portions of the heel cup may hold the vertical axis of the calcaneous essentially coaxial with the axis of the leg. The longitudinal axis of the heel cup and midfoot portions are oriented toward the fifth metatarsus of the foot so as to likewise orient the calcaneous. The midfoot portion has a curvilinear upper bearing surface to support the subtalar. The upper surface of the orthopedic intermediary support member is contoured to engage and cradle the plantar surface of a person's foot, and the bottom surface may be generally flat and planar, or shaped to conform to the inner surface of a shoe.

The heel cup portion permits limited freedom of movement of the heel relative to the midfoot portion when the orthopedic intermediary support member is worn. The bottom region of the heel cup may be thicker to absorb the primary force of a heel strike. Reinforcement support members may optionally be embedded and secured into the heel cup to provide additional support for the calcaneous. Regions of softer, pliable material or detents may be formed in the bottom region of the heel cup to provide comfort and relief from heel spurs, for example, or atrophy of the fat pad.

FIG. 4b shows a perspective view of an orthopedic intermediary support member, in accordance with one embodiment of the present invention. Orthopedic intermediary support member 420 includes a cupped heel portion 430 with an upper bearing surface 432 and a midfoot portion 434 having a medial longitudinal arch support 436 and a curvilinear upper bearing surface 438 for engaging an arch portion of the foot. Cupped heel portion 430 includes a heel cup angle alpha (α) corresponding to the angle between a line essentially parallel to lower bearing surface 422 of cupped heel portion 430 and a line essentially tangential to the top of the outer arcuate portion of cupped heel portion 430, with a larger heel cup angle corresponding to a fuller heel cup.

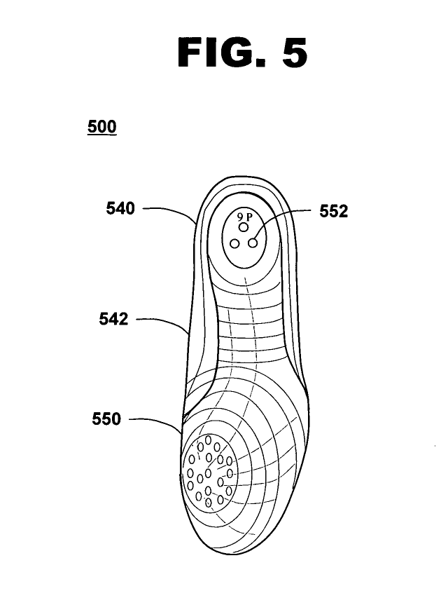

FIG. 5 illustrates a bottom view of an exchangeable innersole 540 for a pronation condition, in accordance with one embodiment of the present invention at 500. Pronation innersole 542 mates with an orthopedic intermediary support member affixed to a shoe to provide a user-selected orthopedic correction factor such as a pronation correction factor. Pronation innersole 542 is insertable into the shoe by the user. The lower bearing surface of pronation innersole 542 is shaped to substantially conform to upper bearing surfaces of the orthopedic intermediary support member and to the inner surfaces of a shoe distal to an anterior end of the orthopedic intermediary support member. Pronation innersole 542 is adapted to provide additional support in the medial longitudinal arch region of the foot to provide a pronation correction factor. A thin, absorptive layer 550 may be disposed on an upper bearing surface of pronation innersole 542 to provide additional comfort for the user. Pronation innersole 542 may include one or more protrusions 552 on a lower bearing surface of pronation innersole 542 to lock into corresponding recesses on an upper bearing surface of an orthopedic intermediary support member. Protrusions 552 may be located, for example, in a region under the heel of a foot.

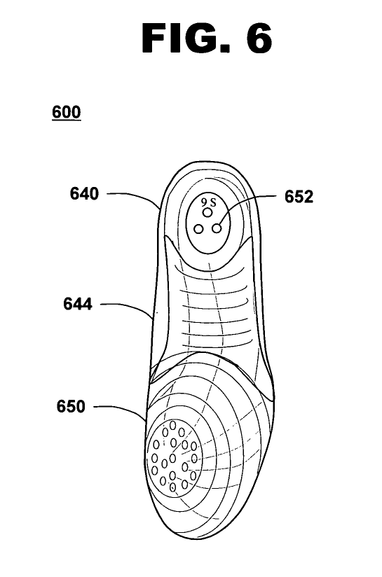

FIG. 6 illustrates a bottom view of an exchangeable innersole 640 for a supination condition, in accordance with one embodiment of the present invention at 600. Supination innersole 644 mates with an orthopedic intermediary support member affixed to a shoe to provide a user-selected orthopedic correction factor such as a supination correction factor. Supination innersole 644 is insertable into the shoe by the user. The lower bearing surface of supination innersole 644 is shaped to substantially conform to upper bearing surfaces of the orthopedic intermediary support member and to the inner surfaces of a shoe distal to an anterior end of the orthopedic intermediary support member. Supination innersole 644 is adapted to provide additional support in the medial longitudinal arch region of the foot to provide a supination correction factor. A thin, absorptive layer 650 may be disposed on an upper bearing surface of exchangeable innersole 640 to provide additional comfort for the user. Supination innersole 644 may include one or more protrusions 652 on a lower bearing surface of supination innersole 644 to lock into corresponding recesses on an upper bearing surface of an orthopedic intermediary support member. Protrusions 652 may be located, for example, in a region under the heel of a foot.

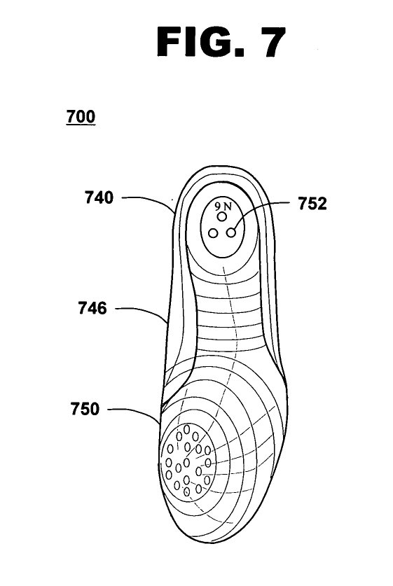

FIG. 7 illustrates a bottom view of an exchangeable innersole 740 for a neutral condition, in accordance with one embodiment of the present invention at 700. Neutral innersole 746 mates with an orthopedic intermediary support member affixed to a shoe to provide a user-selected orthopedic correction factor such as a neutral correction factor. Neutral innersole 746 is insertable into the shoe by the user. The lower bearing surface of neutral innersole 746 is shaped to substantially conform to upper bearing surfaces of the orthopedic intermediary support member and to the inner surfaces of a shoe distal to an anterior end of the orthopedic intermediary support member. Neutral innersole 746 is adapted to provide nominal support in the medial longitudinal arch region of the foot to provide a neutral correction factor. A thin, absorptive layer 750 may be disposed on an upper bearing surface of neutral innersole 746 to provide additional comfort for the user. Neutral innersole 746 may include one or more protrusions 752 on a lower bearing surface of neutral innersole 746 to lock into corresponding recesses on an upper bearing surface of an orthopedic intermediary support member. Protrusions 752 may be located, for example, in a region under the heel of a foot.

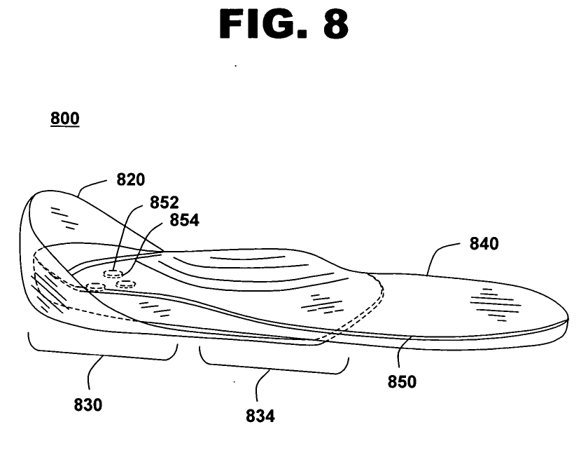

FIG. 8 illustrates a perspective view of an orthopedic intermediary support member mated with an exchangeable innersole, in accordance with one embodiment of the present invention at 800. An orthopedic intermediary support member 820 includes a cupped heel portion 830 and a midfoot portion 834. A user-selectable exchangeable innersole 840 comprising, for example, one of a pronation innersole, a supination innersole and a neutral innersole, mates with orthopedic intermediary support member 820 to provide a user-selected orthotic correction factor such as a pronation correction factor, a supination correction factor, or a neutral correction factor. Exchangeable innersole 840 may include at least one protrusion 852 on a lower bearing surface of exchangeable innersole 840 that locks into a corresponding recess 854 on the concave upper bearing surface of orthopedic intermediary support member 820. An absorptive layer 850 may be disposed on an upper bearing surface of exchangeable innersole 840.

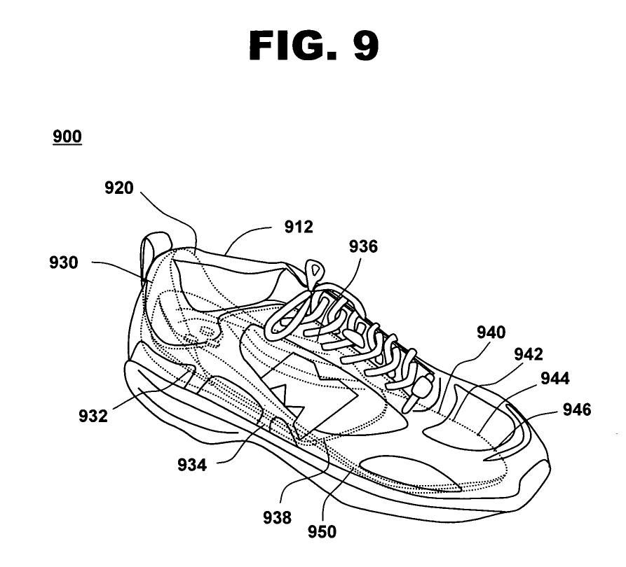

FIG. 9 illustrates a perspective view of an article of footwear with an orthopedic intermediary support member and a user-selectable exchangeable innersole, in accordance with one embodiment of the present invention at 900. Article of footwear 912 comprising, for example, an athletic shoe, includes an orthopedic intermediary support member 920 having a cupped heel portion 930 with a concave upper bearing surface 932 and a midfoot portion 934 with a medial longitudinal arch support 936 and a curvilinear upper bearing surface 938. Orthopedic intermediary support member 920 is fixedly attached to the shoe. An exchangeable innersole 940 with a user-selected orthopedic correction factor is inserted into the shoe to mate with orthopedic intermediary support member 920. Exchangeable innersole 940 comprises, for example, a pronation innersole 942, a supination innersole 944, or a neutral innersole 946 to provide the desired correction factor.

Orthopedic intermediary support member 920 of partial or two-thirds length conforms to the contours of the sole of a wearer's foot during use, having a perimeter that encompasses a foot from the heel to a region near the balls of the foot. A full-length version extends from the heel of a foot to the anterior ends of the toes.

Article of footwear 912 comprises orthopedic intermediary support member 920, wherein orthopedic intermediary support member 920 comprises a cupped heel portion having a concave upper bearing surface that extends above a posterior portion of a heel bone and a midfoot portion with a curvilinear upper bearing surface having a medial longitudinal arch support, the cupped heel portion and the midfoot portion extending approximately two-thirds of the length of a foot. Orthopedic intermediary support member 920 may also include a forefoot portion having a substantially flat upper bearing surface. Orthopedic intermediary support member 920 conforms and is fixedly attached to an inside surface of the shoe. The partial length or full length support member and matching exchangeable innersoles 940 may be readily adapted to different shoe sizes and types such as work shoes, sport shoes, shoes with heels and so forth. An absorptive layer 950 may be disposed on an upper bearing surface of exchangeable innersole 940.

Although an athletic shoe is indicated, the orthopedic intermediary support member may be built into nearly any article of footwear, including a running shoe, a tennis shoe, a cross-trainer shoe, a walking shoe, a children's shoe, a work shoe, a dress shoe, a casual shoe, an open-toe shoe, an orthopedic shoe, a sandal, a military shoe, an all-terrain shoe, a diabetic shoe, a specialty shoe, and a boot. In the case of an athletic shoe and similar shoes, soft-sided uppers may be formed of cloth, vinyl, or other flexible materials that yield outwardly under pressure, thereby providing little inward buttressing around the insole. In the case of a boot, the orthopedic intermediary support member may be integrated into the insole of a work boot, a military boot, or a fashion boot.

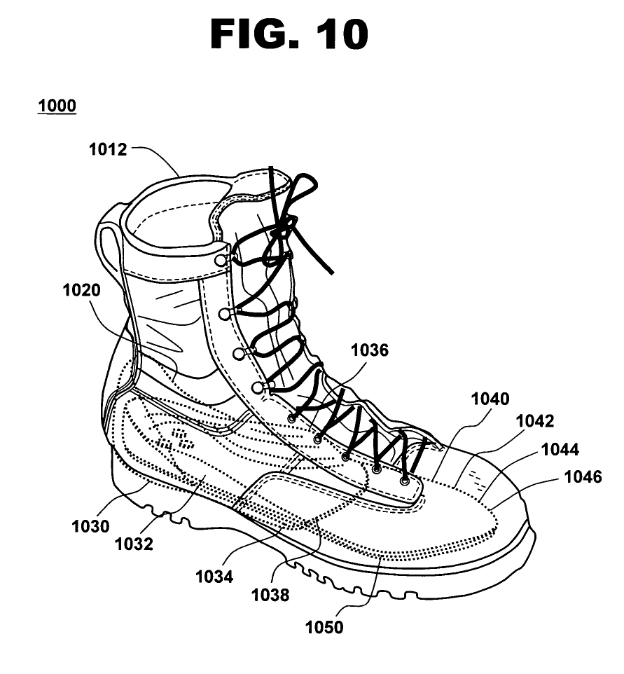

FIG. 10 illustrates a perspective view of an article of footwear with an orthopedic intermediary support member and an exchangeable innersole, in accordance with one embodiment of the present invention at 1000. Article of footwear 1012 comprising, for example, a boot, includes an orthopedic intermediary support member 1020 having a cupped heel portion 1030 with a concave upper bearing surface 1032 and a midfoot portion 1034 with a medial longitudinal arch support 1036 and a curvilinear upper bearing surface 1038. Orthopedic intermediary support member 1020 is fixedly attached to the shoe. An exchangeable innersole 1040 with a user-selected orthopedic correction factor is inserted into the shoe to mate with orthopedic intermediary support member 1020. Exchangeable innersole 1040 comprises, for example, a pronation innersole 1042, a supination innersole 1044, or a neutral innersole 1046 to provide the desired correction factor.

As in other embodiments of the present invention, orthopedic intermediary support member 1020 may have a flat portion corresponding to the forefoot, a midfoot portion 1034 extending from the medial side of the foot to the lateral side of the foot with a generally curvilinear upper bearing surface to support the arches, and a concave side wall portion formed continuously therewith, extending rearwards from the midfoot portion to an area corresponding to the heel of the foot and extending upwardly to engage the medial, posterior and lateral sides of the heel. A partial or full-length orthopedic intermediary support member 1020 may be built directly into the boot or attached to the inside of the boot. An absorptive layer 1050 may be disposed on and attached to an upper bearing surface of exchangeable innersole 1040.

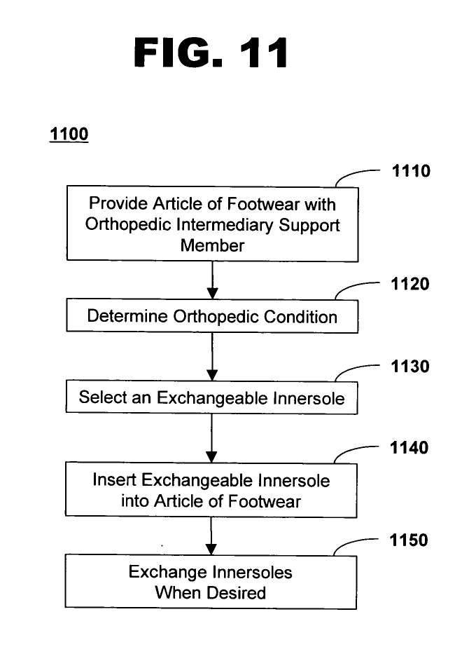

FIG. 11 illustrates a flow diagram of a method for using a multi-component orthopedic system, in accordance with one embodiment of the present invention at 1100. Multi-component orthopedic system use method 1100 includes steps to use a multi-innersole component system.

An article of footwear with an orthopedic intermediary support member is provided, as seen at block 1110. The orthopedic intermediary support member including a cupped heel portion and a midfoot portion is fixedly attached to the article of footwear.

An orthopedic condition of a user is determined, as seen at block 1120. The orthopedic condition, such as a pronation condition, a supination condition or a neutral condition, may be determined by the user, a podiatrist, or other foot specialist.

One innersole from a set of exchangeable innersoles is selected, as seen at block 1130. The set of exchangeable innersoles may include, for example, a pronation innersole, a supination innersole, and a neutral innersole. The exchangeable innersole mates with the orthopedic intermediary support member to provide an orthopedic correction factor such as a pronation correction factor, a supination correction factor, and a neutral correction factor.

The selected exchangeable innersole is inserted into the article of footwear, as seen at block 1140. The exchangeable innersole is inserted, for example, by positioning the article of footwear and pushing the exchangeable innersole into the shoe or boot until the exchangeable innersole fits firmly and mates with the orthopedic intermediary support member. If locking features are included on the exchangeable innersole and the orthopedic intermediary support member, then the protruding features and the corresponding recessed features are appropriately engaged.

The selected exchangeable innersole may be exchanged with a second exchangeable innersole based on the orthopedic condition of the user, as seen at block 1150. For example, when a user has initially selected an incorrect correction factor or has experienced therapeutic improvements after using the first exchangeable innersole for a period of time, the first exchangeable innersole is removed from within the article of footwear and the second exchangeable innersole is inserted into the article of footwear and mated with the orthopedic intermediary support member to provide a second orthopedic correction factor.

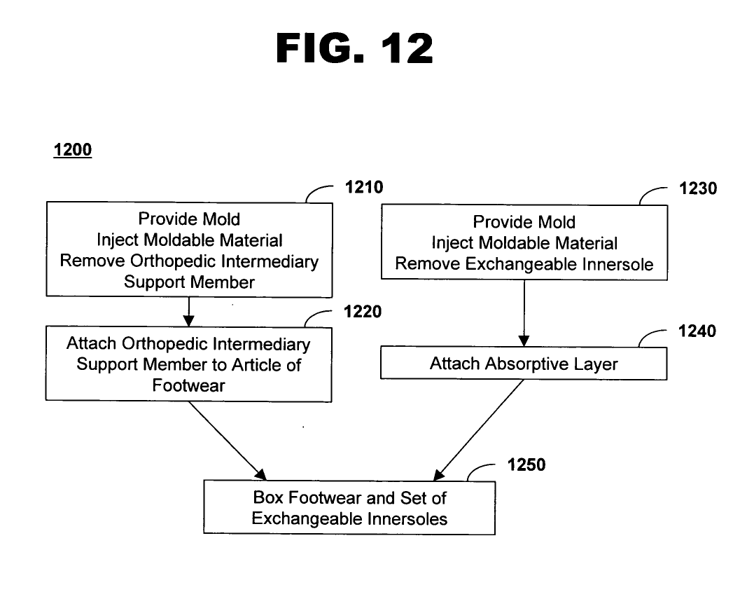

FIG. 12 illustrates a flow diagram of a method of manufacturing an orthopedic intermediary support member and an exchangeable innersole for a shoe, in accordance with the present invention at 1200.

Orthopedic intermediary support member and exchangeable innersole manufacturing method 1200 begins by providing an orthopedic intermediary support member mold, as seen at block 1210. The mold has a cavity for a cupped heel portion, a midfoot portion and optionally a forefoot portion. The cupped heel portion has an upwardly concave upper bearing surface, and the midfoot portion has a medial longitudinal arch support with a curvilinear upper bearing surface. The forefoot portion may have a substantially flat upper bearing surface and outline the distal end of the shoe or boot in which it may be inserted. The mold may have features for recesses in the orthopedic intermediary support member to mate and lock with a corresponding protrusion on the lower bearing surface of the exchangeable innersole. The mold may have additional features such as holes to lighten the orthopedic intermediary support member or texture to aid in gripping the heel of the foot.

An injection-molding compound is injected into the orthopedic intermediary support member mold. The injection-molding compound may include a neoprene rubber, a silicone rubber, an elastomer, a polymeric material, a urethane, polyethylene teraphthalate, a viscoelastic material, a silicone gel, and combinations thereof. The compound may be cured or treated to form the flexible material, as is known in the art. Additional pre-molded support material, such as a more rigid reinforcement mesh, may be provided and inserted into the orthopedic intermediary support member mold prior to injecting the injection-molding compound. When completed, the orthopedic intermediary support member is released and removed from the mold.

When removed, the orthopedic intermediary support member is attached to the article of footwear, as seen at block 1220. For example, the orthopedic intermediary support member may be attached to the inner surface of the shoe or boot using glue, adhesives, stitching, tacking, or other fixation techniques known in the art.

An exchangeable innersole mold is provided, as seen at block 1230. The mold has a cavity corresponding to the shape of the exchangeable innersole, such that the exchangeable innersole may mate with an orthopedic support member. The mold includes features to provide orthopedic correction factors such as pronation, supination and neutral. The mold may have features for protrusions on the lower bearing surface of the exchangeable innersole to mate and lock into corresponding recesses on the upper bearing surface of the orthopedic intermediary support member. The mold may have additional features such as texture to aid engagement between the exchangeable innersole and the orthopedic intermediary support member, indicia, or other markings.

An injection-molding compound is injected into the exchangeable innersole mold. The injection-molding compound may be a lightweight and durable thermoplastic such as polyethylene or cross-linked ethylene vinyl acetate foam, cross-linked polyethylene, poly(ethylene-vinyl acetate), polyvinyl chloride, an acrylic, synthetic and natural latex rubbers, block polymer elastomers, thermoplastic elastomers, polystyrene, ethylenepropolene rubbers, silicone elastomers, polystyrene, polyurea or polyurethane, a polyurethane foam, an elastomeric foam, a moldable foam, a non-foam elastomer, a silicone gel, a viscoelastic material, and combinations thereof. The compound may be cured or treated to form the flexible material, as is known in the art. When completed, the exchangeable innersole is released and removed from the mold.

Alternatively, the exchangeable innersole may be cut from sheets or pieces of elastomeric foam or other suitable materials, and layered with other pieces to provide the desired orthopedic correction factor.

An absorbing material optionally may be attached to the exchangeable innersole, as seen at block 1240. For example, a sheet of absorptive material may be cut and glued to the upper bearing surface of the exchangeable innersole.

The article of footwear with the orthopedic intermediary support member and a set of exchangeable innersoles may be boxed, as seen at block 1250. The box may be marked with the size of the shoe or boot and an indicator of inclusion of the multi-innersole component system with one or more exchangeable innersoles, and shipped for distribution and sale. The multi-component orthopedic system provides a more comfortable and supportive shoe due to the ability to accommodate different foot types at user discretion.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

What is claimed is:

1. An innersole system for an article of footwear, comprising:
an orthopedic intermediary support member including a cupped heel portion having a concave upper bearing surface and a midfoot portion having a medial longitudinal arch support with a curvilinear upper bearing surface; and
a user-selectable exchangeable innersole; wherein the exchangeable innersole mates with the orthopedic intermediary support member to provide a user-selected orthopedic correction factor.

2. The innersole system of claim 1 wherein the cupped heel portion extends above a most posterior cephalad portion of a calcaneous.

3. The innersole system of claim 1 wherein the cupped heel portion is continuously coupled to the midfoot portion.

4. The innersole system of claim 1 wherein the orthopedic intermediary support member is fixedly attached to the article of footwear.

5. The innersole system of claim 1 wherein the exchangeable innersole is selected from the group consisting of a pronation innersole, a supination innersole, and a neutral innersole.

6. The innersole system of claim 1 wherein the user-selected orthopedic correction factor comprises one of a pronation correction factor, a supination correction factor, and a neutral correction factor.

7. The innersole system of claim 1 wherein the cupped heel portion and the midfoot portion of the orthopedic intermediary support member cooperate with the exchangeable innersole to invert a subtalor joint of a foot to a position of slight inversion to lock a midtarsal joint during ambulation of the foot.

8. The innersole system of claim 1 wherein the concave upper bearing surface of the cupped heel portion comprises a heel cup angle of at least 60 degrees.

9. The innersole system of claim 1 wherein the midfoot portion of the orthopedic intermediary support member extends from the cupped heel portion to an opposite end corresponding to an anterior end of a metatarsal bone.

10. The innersole system of claim 1 wherein a lower bearing surface of the exchangeable innersole is contoured to conformably mate with the concave upper bearing surface and the curvilinear upper bearing surface of the orthopedic intermediary support member.

11. The innersole system of claim 1 wherein the exchangeable innersole includes at least one protrusion on a lower bearing surface of the exchangeable innersole to lock into a corresponding recess on the concave upper bearing surface of the orthopedic intermediary support member.

12. The innersole system of claim 1 wherein the article of footwear is selected from the group consisting of an athletic shoe, a running shoe, a tennis shoe, a cross-trainer shoe, a walking shoe, a children's shoe, a work shoe, a dress shoe, a casual shoe, an open-toe shoe, an orthopedic shoe, a sandal, a military shoe, an all-terrain shoe, a diabetic shoe, a specialty shoe, and a boot.

13. The innersole system of claim 1 further comprising:
an absorptive layer disposed on an upper bearing surface of the exchangeable innersole.

14. An article of footwear, comprising:
an orthopedic intermediary support member fixedly attached to the article of footwear, the orthopedic intermediary support member including a cupped heel portion having a concave upper bearing surface and a midfoot portion having a medial longitudinal arch support with a curvilinear upper bearing surface; and
a user-selectable exchangeable innersole; wherein the exchangeable innersole mates with the orthopedic intermediary support member to provide a user-selected orthopedic correction factor.

15. The article of footwear of claim 14 wherein the article of footwear is selected from the group consisting of an athletic shoe, a running shoe, a tennis shoe, a cross-trainer shoe, a walking shoe, a children's shoe, a work shoe, a dress shoe, a casual shoe, an open-toe shoe, an orthopedic shoe, a sandal, a military shoe, an all-terrain shoe, a diabetic shoe, a specialty shoe, and a boot.

16. The article of footwear of claim 14 further comprising:
an absorptive layer disposed on an upper bearing surface of the exchangeable innersole.

17. A method of using a multi-component orthopedic system, comprising:
providing an article of footwear having an orthopedic intermediary support member fixedly attached to the article of footwear;
determining an orthopedic condition of a user;
selecting one of a set of exchangeable innersoles, wherein the exchangeable innersole mates with the orthopedic intermediary support member to provide an orthopedic correction factor; and
inserting the selected exchangeable innersole into the article of footwear.

18. The method of claim 17 wherein the set of exchangeable innersoles includes a pronation innersole, a supination innersole, and a neutral innersole.

19. The method of claim 17 further comprising:
exchanging the selected exchangeable innersole with a second exchangeable innersole based on the orthopedic condition of the user.

20. The method of claim 19 wherein exchanging the selected exchangeable innersole with second exchangeable innersole comprises removing the selected exchangeable innersole from the article of footwear, and inserting the second exchangeable innersole into the article of footwear.
